(19) United States
(12) Patent Application Publication
(10) Pub. No.: US 2019/0092756 A1
(43) Pub. Date: Mar. 28, 2019

(54) CRYSTALLINE FORM A OF 2-[(2R)-2-METHYL-2-PYRROLIDYL]-1H-BENZIMIDAZOLE-7-CARBOXAMIDE DIHYDROCHLORIDE AND PREPARATION METHOD THEREOF

(71) Applicant: Crystal Pharmatech Co., Ltd., Suzhou, Jiangsu (CN)

(72) Inventors: Minhua Chen, Suzhou (CN); Yanfeng Zhang, Suzhou (CN); Kai Liu, Suzhou (CN); Xiaoyu Zhang, Zuzhou (CN)

(21) Appl. No.: 16/082,830

(22) PCT Filed: Mar. 8, 2017
(86) PCT No.: PCT/CN2017/075990

§ 371 Date: Sep. 6, 2018

(30) Foreign Application Priority Data
Sep. 3, 2016 (CN) 201610132878.X

(51) Int. Cl. C07D 403/04; A61P 35/04

(52) U.S. Cl. CPC C07D 403/04; A61P 35/04

(57) ABSTRACT

Provided are a crystalline form A of 2-[(2R)-2-methyl-2-pyrrolidyl]-1H-benzimidazole-7-carboxamide dihydrochloride and the preparation method and use thereof. The X-ray powder diffraction pattern of crystalline form A shows characteristic peaks at 2theta values of 8.3°±0.2°, 26.7°±0.2°, 16.1°±0.2°. Crystalline form A, compared with the existing crystalline forms, has a surprisingly excellent solubility, mechanical and storage stabilities and particle size distribution, is a more ideal crystalline fotbnn compared with the prior art forms, better satisfies medicinal requirements, and plays an important role in future optimization and development of the drug.

### TECHNICAL FIELD

The present disclosure relates to the field of pharmaceutical chemistry, particularly in relation to novel crystalline form A of ABT-888 dihydrochloride as well as the preparation method and use thereof.

### BACKGROUND

2-[(2R)-2-methyl-2-pyrrolidinyl]-1H-benzimidazole-7-carboxamide (shown as formula I), also 10 named as ABT-888 (Veliparib), is a novel high-selection polyadenosine diphosphate-ribose polymerase (PARP) inhibitor developed by Abbvie. In-vivo and in-vitro experiments show that ABT-888 has remarkable effect on inhibiting PARP activity and treating metastatic breast cancer, colon cancer, metastatic melanoma and brain tumors. At present, ABT-888 in combination with all-brain radiotherapy can be used for treating metastatic brain tumors, metastatic breast cancer, colon cancer and metastatic melanoma. The combination of ABT-888 and Temozolomide is in clinical study for treating breast cancer.

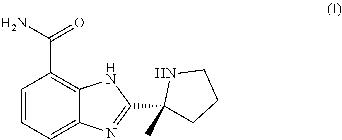 [SMILES: C[C@]1(c2nc3cccc(C(N)=O)c3[nH]2)CCCN1]

It is generally known that crystalline forms greatly affect drug's quality. Different crystalline forms may have remarkable difference in appearance, solubility, melting point, dissolution profile and so on, thus affect drug's stability, bioavailability and efficacy. Therefore, it is of great significance to develop novel and more suitable crystalline forms for drug development.

As is known to the skilled in the art, the presence of new solid polymorphs of a known chemical substance solid is unpredictable. The existence or the number of the polymorphs is also unpredictable. In addition, it is also unpredictable that under what conditions a specific form will crystallize, and what are the characteristics of the polymorphic form. Since different polymorphs have different properties (e.g., solubility, stability), their performances on drug's use and storage are different, it is necessary to study all solid forms, including all polymorphs to provide drugs with improved stability or solubility.

CN101821270A and CN101821269B disclosed two crystalline forms of ABT-888 freebase, namely crystalline form 1 and crystalline form 2, and the preparation methods thereof. According to the aforementioned disclosure, the X-ray powder diffraction of crystalline form 1 shows peaks at 2theta values of about 9.9°, 11.0° and 11.8° and one or more than one additional peaks having respective 2theta values of about 14.6°, 15.2°, 18.2°, 19.6°, 20.3°, 21.3°, 22.5°, 22.8°, 24.7°, 28.5° and 29.1°. The melting point of crystalline form 1 is 188.6±0.8° C. The X-ray powder diffraction of crystalline form 2 shows peaks at 2theta values of about 13.4°, 17.1°, 21.6°, 21.9°, 24.1°, 24.7°, 26.9°, 27.3°, 27.8°, 30.3°, 32.4° and 34.2° and without peaks below about 11.5°. Crystalline form 1 has better solubility, but it is obtained by reacting a monoacid or diacid salt of ABT-888 with a base, crystallizing or recrystallizing from a solid, semisolid, wax or oil of ABT-888 that is mixed with one or more than one solvent through deprotonation reaction. The preparation method of crystalline form 1 is complicated and has high operation requirements. The preparation method of crystalline form 2 is relatively simple by dissolving ABT-888 into methanol solvent, then concentrating at 35° C. and drying to constant weight. However, crystalline form 2 has poor solubility.

The applicant of present disclosure surprisingly discovered a new crystalline form B of ABT-888 freebase in previous researches and has filed a patent application CN105130961A. Compared with crystalline form 1 and form 2 mentioned above, crystalline form B has simple preparation method, and has higher solubility (in a specific embodiment, crystalline form B has solubility of 6.0 mg/mL after 24 hours in SGF). Moreover, crystalline form B has good stability.

To conclude, the crystalline forms of ABT-888 disclosed in prior art are all crystalline forms of free base, and no crystalline form of ABT-888 pharmaceutically acceptable salt was disclosed. Theoretically, suitable salts of a drug can improve its solubility, physical and chemical stabilities. The salt-formation of a drug can improve its physical properties such as melting point, hygroscopicity, crystalline forms and the like, which is important for developing drug's formulation. However, with respect to a specific compound, it is uncertain and unpredictable if there exist suitable salts of a drug, and what are the characteristics of the salts. Therefore, developing a pharmaceutically acceptable salt of ABT-888 with ideal physical properties will have great value for drug's production and application.

### SUMMARY OF THE DISCLOSURE

One objective of the present disclosure is to provide crystalline form A of ABT-888 dihydrochloride, which is an ideal pharmaceutically acceptable salt of ABT-888.

Another objective of the present disclosure is to provide the preparation method and use of above crystalline form A of ABT-888 dihydrochloride.

To achieve the above objectives, the present disclosure uses technical solution as follows:

The X-ray powder diffraction pattern of crystalline form A of formula I compound dihydrochloride (also named crystalline form A of ABT-888 dihydrochloride or briefly crystalline form A) shows characteristic peaks at 2theta values of 8.3°±0.2°, 26.7°±0.2°, and 16.1°±0.2°.

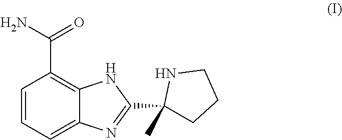 [SMILES: C[C@]1(c2nc3cccc(C(N)=O)c3[nH]2)CCCN1]

According to a further embodiment, the X-ray powder diffraction pattern of crystalline form A of ABT-888 dihydrochloride further shows one or more characteristic peaks at 2theta values of 22.8°±0.2°, 21.3°±0.2°, 15.6°±0.2°, 19.8°±0.2°, and 28.1°±0.2°.

According to a specific and preferred embodiment, the X-ray powder diffraction pattern of crystalline form A of ABT-888 dihydrochloride shows one or more characteristic peaks at 2theta values of 8.3°±0.2°, 26.7°±0.2°, 16.1°±0.2°, 22.8°±0.2°, 21.3°±0.2°, and 15.6°±0.2°.

According to another specific and preferred embodiment, the X-ray powder diffraction pattern of crystalline form A of ABT-888 dihydrochloride shows characteristic peaks at 2theta values of 8.3°±0.2°, 26.7°±0.2°, 16.1°±0.2°, 22.8°±0.2°, 21.3°±0.2°, 15.60°±0.2°, 19.8°±0.2°, and 28.1°±0.2°.

Another objective of the present disclosure is to provide a preparation method of the crystalline form A of ABT-888 dihydrochloride, the method comprises:

1) adding formula I compound into a solvent system selected from alcohols, ketones, esters and nitriles, then stirring to react with hydrochloric acid to obtain a suspension containing formula I compound dihydrochloride;

2) separating the suspension in step 1) to get the solid, and drying to obtain the crystalline Form A.

Furthermore, the said alcohols include, but not limited to ethanol and isopropanol. Said ketones include, but not limited to acetone and methyl isobutyl ketone. Said esters include, but not limited to ethyl acetate and isopropyl acetate. Said nitriles include, but not limited to acetonitrile.

Crystalline form A of ABT-888 dihydrochloride in the present disclosure has better physical properties compared with crystalline forms of ABT-888 freebase. It is more suitable for preparing pharmaceutical compositions/drugs for certain use. Said certain use includes but not limited to the use for the treatment of metastatic breast cancer, colon cancer, metastatic melanoma and brain tumors.

Another objective of the present disclosure is to provide a pharmaceutical composition, wherein the said pharmaceutical composition comprises a therapeutically effective amount of crystalline form A of ABT-888 dihydrochloride in the present disclosure and pharmaceutical excipient.

Another objective of the present disclosure is to provide a method for treating diseases like metastatic breast cancer, colon cancer, metastatic melanoma, brain tumors and so on, and the method comprises administering to patients in need a therapeutically effective amount of crystalline form A of ABT-888 dihydrochloride in the present disclosure.

Due to the implement of the above embodiment, the present disclosure has the following advantages compared with the prior art:

Crystalline form A of ABT-888 dihydrochloride in the present disclosure has unexpected excellent solubility, mechanical and physical stability, and particle size distribution. It is a more ideal crystalline form compared with what has already been disclosed.

### BRIEF DESCRIPTION OF THE DRAWINGS

FIG. 1 shows an XRPD pattern of crystalline form A obtained in example 1.

FIG. 2 shows a DSC curve of crystalline form A obtained in example 1.

FIG. 3 shows a TGA curve of crystalline form A obtained in example 1.

FIG. 4 shows a ¹H NMR spectrum of crystalline form A.

FIG. 5 shows an XRPD pattern of crystalline form A obtained in example 2.

FIG. 6 shows an XRPD pattern of crystalline form A obtained in example 3.

FIG. 7 shows a TGA curve of crystalline form A obtained in example 3.

FIG. 8 shows an XRPD pattern of crystalline form A obtained in example 4.

FIG. 9 shows an XRPD overlay pattern of crystalline form A before and after grinding, the pattern above is before grinding and the below one is after grinding.

FIG. 10 shows an XRPD overlay pattern of crystalline form A before and after storage at 25° C./60% RH and 40° C./75% RH for 1 month. The top, middle and bottom patterns represent initial crystalline form A, crystalline form A after storage at 25° C./60% RH for 1 month and crystalline form A after storage at 40° C./75% RH for 1 month, respectively.

### DETAILED DESCRIPTION OF THE DISCLOSURE

The X-ray powder diffraction pattern of crystalline form A of ABT-888 dihydrochloride in the present disclosure shows characteristic peaks at 2theta values of 8.3°±0.2°, 26.7°±0.2°, and 16.1°±0.2°. The X-ray powder diffraction pattern of crystalline form A further shows one or more characteristic peaks at 2theta values of 22.8°±0.2°, 21.3°±0.2°, 15.6°±0.2°, 19.8°±0.2°, and 28.1°±0.2°. Preferably, the X-ray powder diffraction pattern of crystalline form A shows at least five characteristic peaks mentioned above. More preferably, the X-ray powder diffraction pattern of crystalline form A shows at least six characteristic peaks mentioned above. Further preferably, the X-ray powder diffraction pattern of crystalline form A shows at least seven characteristic peaks mentioned above. Most preferably, the X-ray powder diffraction pattern of crystalline form A shows at least eight characteristic peaks mentioned above.

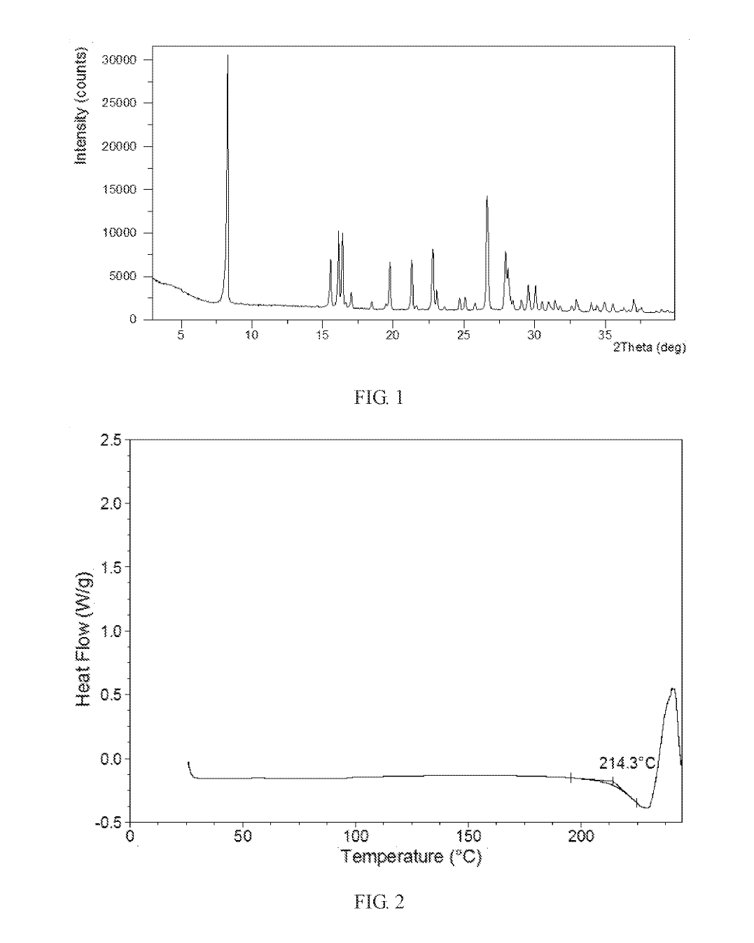

According to a specific embodiment, when performing the differential scanning calorimetry, crystalline form A shows an endothermic peak when heated to around 214° C. (onset temperature). The differential scanning calorimetry curve (DSC curve) is substantially depicted in FIG. 2.

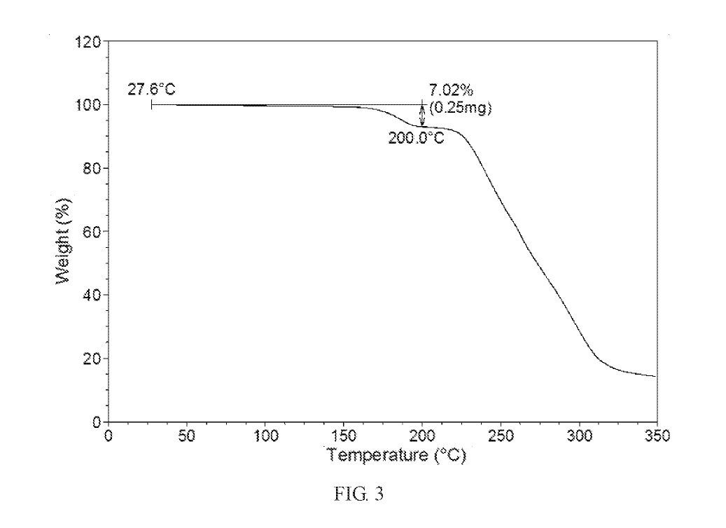

According to another specific embodiment, when performing the thermogravimetric analysis, crystalline form A shows 7.0% weight loss when heated to around 200° C. The thermogravimetric analysis curve (TGA curve) is substantially depicted in FIG. 3.

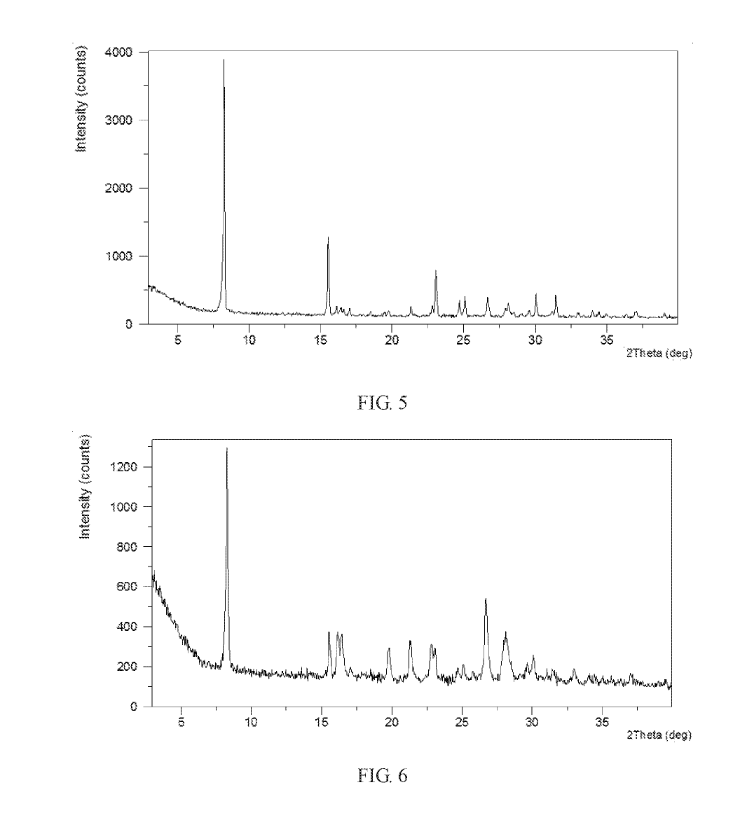

In some specific embodiments of the present disclosure, the X-ray powder diffraction patterns of crystalline form A are substantially depicted in FIG. 1, FIG. 5, FIG. 6 and FIG. 8.

Crystalline form A in the present disclosure has unexpected excellent solubility, good physical and mechanical stability, and its particle size distribution is suitable for drug production. Compared with existing crystalline forms in prior art, it is more suitable for industrial production and application.

In some embodiments, crystalline form A of the present disclosure is pure, single and substantially free of any other crystalline forms. In the present disclosure, the term “substantially free” when used to describe a novel crystalline form, it means that the content of other crystalline forms in the novel crystalline form is less than 20% (w/w), specifically less than 10% (w/w), more specifically less than 5% (w/w) and further more specifically less than 1% (w/w).

The present disclosure provides the preparation method of crystalline form A, the method comprises: 1) adding formula I compound into a solvent system selected from alcohols, ketones, esters and nitriles, then stirring to react with hydrochloric acid to obtain a suspension containing formula I compound dihydrochloride, 2) separating the suspension in step 1) to get the solid, and drying to obtain the crystalline form A.

Furthermore, said alcohols include, but not limited to ethanol and isopropanol. Said ketones include, but not limited to acetone and methyl isobutyl ketone. Said esters include, but not limited to ethyl acetate and isopropyl acetate. Said nitriles include, but not limited to acetonitrile. Said solvent system can be one or more solvents selected from the above solvents without limitation.

According to a specific aspect of the present disclosure, said solvent system is one or more solvents selected from ethanol, isopropanol, acetone, methyl isobutyl ketone, ethyl acetate, isopropyl acetate, acetonitrile.

As a preferred embodiment of the present disclosure, in step 1), formula I compound is added into the solvent system firstly, and then hydrochloric acid is added dropwise with stirring.

According to the present disclosure, a reaction temperature in step 1) is −20° C. to 50° C. Preferably, the temperature is 0° C. to 30° C. Further preferably, the temperature is 20° C. to 30° C. Most preferably, the temperature is room temperature of about 25° C.

In a specific embodiment of the present disclosure, the reaction temperature in step 1) is room temperature.

According to the present disclosure, after the addition of hydrochloric acid in step 1), stirring for at least 6 hours is preferred, stirring for at least 8 hours is more preferred, and stirring for at least 10 hours is further preferred.

In another specific embodiment of the present disclosure, in step 1), a concentration of the hydrochloric acid is 10-12 mol/L, and a molar ratio of hydrochloric acid and compound formula I is 2-2.1:1, preferably is 2.02-2.06:1.

According to the present disclosure, formula I compound (also named as Veliparib or ABT-888) used as the starting material can be in solid (crystalline or amorphous), semi-solid, waxy or oil forms. Preferably, Veliparib used as starting material is in solid form.

According to the present disclosure, said “stirring” is completed by using a conventional method in the field such as a mechanical stirring or a magnetic stirring and the stirring speed is 50 to 1800 r/min, preferably 300 to 900 r/min.

Unless otherwise specified, said “drying” can be carried out at room temperature or a higher temperature. The drying temperature is from room temperature to about 60° C., preferably from room temperature to about 50° C., more preferably from 30° C. to about 50° C., and further preferably from 30° C. to about 40° C. The drying time can be 2 to 48 hours, or overnight. Drying is carried out in a fume hood, oven or vacuum oven.

In the present disclosure, “crystal” or “crystalline form” refers to the crystal or the crystal form being identified by the X-ray diffraction pattern shown herein. The person skilled in the art are able to understand that physical and chemical properties discussed herein can be characterized and the experimental errors depend on the conditions of instruments, the sample preparations and the purity of samples. In particular, the scientists in this field generally know that the X-ray diffraction pattern usually may change with the change of the experimental conditions. It is necessary to point out that, the relative intensity of the X-ray diffraction pattern is likely to change with the change of the experimental conditions; therefore, the sequence of peak intensity cannot be regarded as the only or the determining factor. Moreover, the experimental errors of the peak angles are 5% or less, so such errors should be considered and generally the allowed errors are ±0.2° 2θ. In addition, due to the effect of the experimental factors including sample height, peak angles may have an overall shifting; generally, certain shifting is allowed. Hence, the scientists in this field may understand that, it is unnecessary that the X-ray diffraction pattern of a crystal form in the present disclosure should be exactly the same with X-ray diffraction patterns of the example shown herein. Any crystal forms whose X-ray diffraction patterns have the same or similar characteristic peaks should be within the scope of the present disclosure. The scientists in this field can compare the patterns shown in the present disclosure with that of an unknown crystal form in order to identify whether these two groups of patterns reflect the same or different crystal forms.

“Crystalline form” and “polymorphic form” as well as other related terms in the present disclosure refer to the solid compounds whose crystal structure is being in a special crystal form state. The difference in the physical and chemical properties of the polymorphic forms may be embodied in physical stability, compressibility, density, dissolution rate, etc. In extreme cases, the difference in solubility or dissolution rate may result in drugs with low efficiency and toxicity.

Crystalline form A of ABT-888 dihydrochloride in the present disclosure has more excellent physical properties compared with crystalline forms of ABT-888 freebase, and thus is more suitable for preparing pharmaceutical compositions/drugs for corresponding use. Said corresponding use includes but not limited to the use for the treatment of metastatic breast cancer, colon cancer, metastatic melanoma and brain tumors.

Another objective of the present disclosure is to provide a pharmaceutical composition comprising a therapeutically effective amount of crystalline form A of ABT-888 dihydrochloride and pharmaceutical excipient. Generally, a therapeutically effective amount of crystalline form A is mixed or contacted with one or more pharmaceutical acceptable excipients to make pharmaceutical composition or formulation, and the pharmaceutical composition or formulation are prepared by well-known method in the pharmaceutical field. According to a specific and preferable embodiment, said pharmaceutical composition is a pharmaceutical formulation.

The term “effective treatment amount” or “therapeutically effective amount” as used herein means that amount of an active compound that elicits the biological or medicinal response in a tissue, system, animal or human that is being sought by a researcher, veterinarian, medical doctor, or other clinician.

As used herein, the term “treatment” refers to one or more of the following: (1) Preventing disease, for example, preventing the disease, illness or disorder in an individual who may be suffering from a disease, illness or disorder but not suffering from or displaying a lesion or symptom of the disease, (2) Inhibiting the disease, for example, inhibiting the disease, illness or disorder in an individual who is suffering from or displaying a lesion or symptom of the disease, illness or disorder, and (3) Improving the disease, for example, improving the disease, illness or disorder in an individual who is suffering from or displaying a lesion or symptom of the disease, illness or disorder (that is to reverse the lesion and/or symptoms), for example, reducing the severity of the disease.

Furthermore, the present disclosure provides a pharmaceutical composition. In addition to the crystalline of the present disclosure, the pharmaceutical compositions can also contain other pharmaceutically acceptable salts, crystalline form or amorphous form of the salt. Optionally, the crystalline forms of the present disclosure may be administered as the sole active agent, or they may be administered in combination with other active agents that have the same or a similar therapeutic activity, and other compounds identified as safe and effective when being administered with the above combination.

It should be noted that the numerical value and the scope of the present disclosure should not be narrowly understood as a value or numerical value range. It should be understood by those skilled in the art that the specific numerical value can be floated according to the specific technical environment on the basis that the spirit and principle of the disclosure are not depart from the spirit and principle of the disclosure. In the present disclosure, the number of floating ranges which can be expected by one of skilled in the art is represented by the term “about”.

The present disclosure will be further explained by the specific embodiments and the specific embodiments are not intended to limit the scope of the present disclosure. The skilled in the art can make improvements to the process of preparation and the used instruments within the scope of the claims, and those improvements should be considered as falling into the scope of the present disclosure. Therefore, the protective scope of the present disclosure patent should be defined by the claims.

In the following examples, the test method is generally implemented according to a conventional condition or a condition that manufacturer recommends.

The abbreviations used in the disclosure are explained as follows:

XRPD: X-ray Powder Diffraction

DSC: Differential Scanning Calorimetry

TGA: Thermogravimetric Analysis

¹H NMR: ¹H Nuclear Magnetic Resonance

Acetonitrile: ACN

Ethanol: EtOH

Hydrochloric acid: HCl

Ethyl acetate: EtOAc

X-ray powder diffraction pattern in the present disclosure was acquired by a Panalytical Empyrean X-ray powder diffractometer. The parameters of the X-ray powder diffraction method of the present disclosure were as follows:

X-ray Reflection: Cu, Kα

Kα1 (Å): 1.540598; Kα2 (Å): 1.544426

Kα2/Kα1 intensity ratio: 0.50

Voltage: 45 (kV)

Current: 40 (mA)

Scan range: from 3.0 degree to 40.0 degree

The data of differential scanning calorimetry (DSC) was acquired by TA Instruments Q2000 MDSC, with Thermal Advantage as instrument control software and Universal Analysis as analysis software. Generally, 1-10 mg sample is put into an aluminum crucible (unless otherwise specified, the aluminum crucible is covered). The temperature of sample was raised from room temperature to 250° C. with heating rate of 10° C./min under the protection of dry nitrogen with flow rate of 50 mL/min, while the TA software records the heat change of the sample during the heating process.

The data of thermogravimetric analysis (TGA) was acquired by TA Instruments Q5000 TGA, with Thermal Advantage as instrument control software and Universal Analysis as analysis software. Generally, 5˜15 mg sample is put into a platinum crucible. With segmented high resolution detection, the temperature of sample was raised from room temperature to 350° C. with heating rate of 10° C./min under the protection of dry nitrogen with flow rate of 50 mL/min, while the TA software records the weight change of the sample during the heating process.

The data of ¹H nuclear magnetic resonance (¹H NMR) was acquired by the nuclear magnetic resonance spectrometer of Bruker Avance II DMX 400M HZ. Dissolve 1-5 mg sample into 0.5 mL deuterated dimethyl sulfoxide solvent to prepare a solution with concentration of 2-10 mg/mL.

The result of particle size distribution (PSD) in the present disclosure was acquired by laser particle size analyzer with S3500 model from Microtrac company. The Microtrac S3500 is equipped with a SDC (Sample Delivery Controller) sampling system. This experiment uses a wet method and the dispersion medium is Isopar G. The method and parameters of the laser particle size analyzer are as follows:

| | |
|---|---|
| Size distribution: Volume distribution | Run time: 10 s |
| Dispersion medium: Isopar G | Particle coordinates: Standard |
| Run number: 3 | Disperse medium refractive |
| | index: 1.42 |
| Transparency: Transparent | Residual: Enabled |
| Particle refractive index: 1.5 | Flow rate: 60%* |
| Particle shape: Irregular | Filtration: Enabled |
| Ultrasonic power: 30 W | Ultrasonic time: 30 s |

*Flow rate 60% is 60% of 65 mL/s.

The abbreviations used in the present disclosure are explained as follows:

MV: Average based on volume

D10: The D10 describes the diameter where 10% of the distribution has a smaller particle size.

D50: The D50 describes the diameter where 50% of the distribution has a smaller particle size. The median is also called D50.

D90: The D90 describes the diameter where 90% of the distribution has a smaller particle size. It should be noted that the reason why Veliparib freebase in solid state is used as starting material in the below examples is that Veliparib freebase in solid state is easier to obtain, and it does not mean that only this solid state form can be used. According to the inventors' experiments, the final crystalline form is closely related to the preparation conditions, regardless of the solid state of the starting material.

### Example 1 Preparation of Crystalline Form A

2.0 g of Veliparib in powder was added into a 20.0 mL glass vial, followed by the addition of 10 mL of ACN. Then aqueous HCl solution (1.4 mL, 12 mol/mL) was dropped while stirring at room temperature (25° C.±2° C.). Keep stirring at room temperature for 12 hours. After being separated by centrifugation and dried under vacuum, the obtained solid sample was analyzed by XRPD, DSC and TGA, of which the crystalline form was confirmed to be crystalline form A.

The XRPD data of crystalline form A obtained in this example are listed in Table 1. The XRPD pattern, DSC curve and TGA curve are displayed in FIG. 1, FIG. 2 and FIG. 3, respectively.

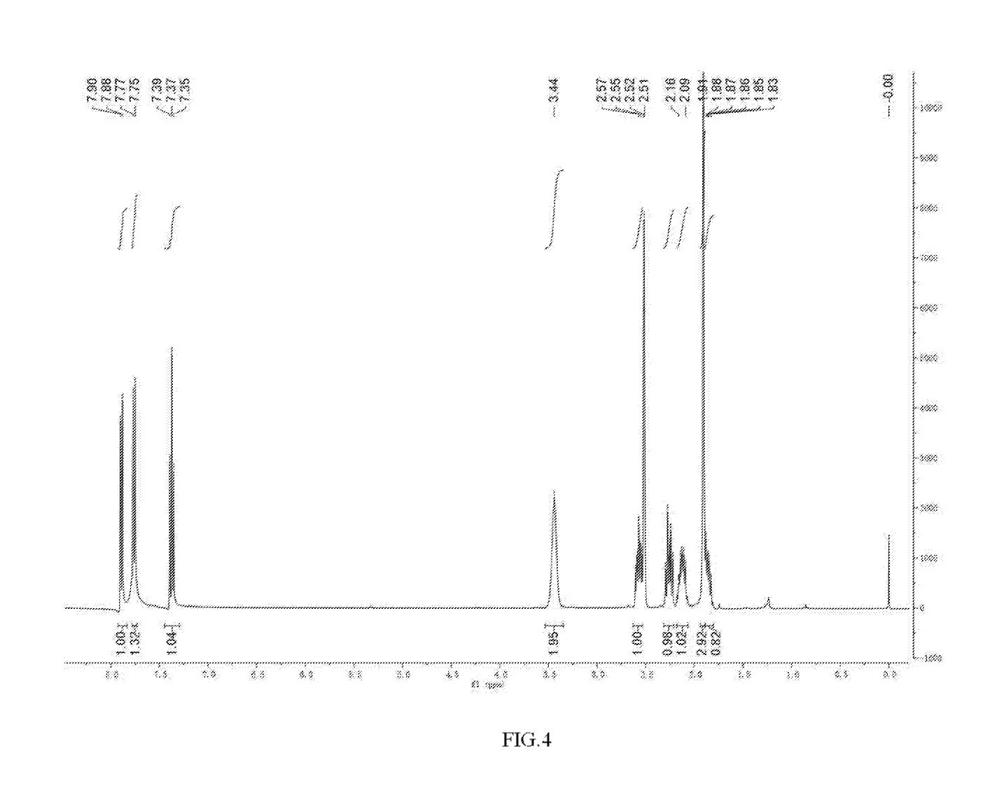

The ¹H NMR spectrum of crystalline form A obtained in this example is displayed in FIG. 4, and the corresponding data are as following: ¹H NMR (400 MHz, DMSO) δ 7.89 (d, J=7.6 Hz, 1H), 7.76 (d, J=7.9 Hz, 1H), 7.37 (t, J=7.8 Hz, 1H), 3.44 (s, 2H), 2.57 (ddd, J=13.2, 8.1, 5.3 Hz, 1H), 2.26 (dt, J=13.1, 7.9 Hz, 1H), 2.13 (ddd, J=13.4, 7.7, 2.2 Hz, 1H), 1.91 (s, 3H), 1.89-1.80 (m, 1H).

TABLE 1

| 2theta | d-spacing | Intensity (%) |
|---|---|---|
| 8.27 | 10.69 | 100.00 |
| 15.55 | 5.70 | 19.86 |
| 16.12 | 5.50 | 31.56 |
| 16.41 | 5.40 | 30.45 |
| 17.02 | 5.21 | 5.91 |
| 19.76 | 4.49 | 19.30 |
| 21.31 | 4.17 | 20.57 |
| 22.79 | 3.90 | 24.88 |
| 23.07 | 3.85 | 8.26 |
| 24.70 | 3.60 | 4.98 |
| 26.65 | 3.34 | 44.80 |
| 27.94 | 3.19 | 24.21 |
| 28.13 | 3.17 | 17.37 |
| 29.56 | 3.02 | 10.90 |
| 30.06 | 2.97 | 10.48 |
| 30.53 | 2.93 | 3.93 |
| 30.99 | 2.89 | 4.02 |
| 31.45 | 2.84 | 4.64 |
| 32.97 | 2.72 | 5.14 |
| 34.00 | 2.64 | 3.83 |
| 34.96 | 2.57 | 4.14 |
| 35.54 | 2.53 | 3.38 |
| 37.02 | 2.43 | 5.36 |

### Example 2 Preparation of Crystalline Form A

10.0 mg of Veliparib in powder was added into a 1.5 mL glass vial, followed by the addition of 0.5 mL of EtOH. Then aqueous HCl solution (7.0 μL, 12 mol/mL) was dropped while stirring at room temperature. Keep stirring at room temperature for 12 hours. After being separated by centrifugation, the obtained solid was analyzed by XRPD, of which the crystal form was confirmed to be crystalline form A.

The XRPD data of crystalline form A obtained in this example are listed in Table 2, and the corresponding XRPD pattern is displayed in FIG. 5.

TABLE 2

| 2theta | d-spacing | Intensity (%) |
|---|---|---|
| 8.27 | 10.69 | 100.00 |
| 15.55 | 5.70 | 30.52 |
| 16.11 | 5.50 | 2.99 |
| 17.03 | 5.21 | 7.27 |
| 19.62 | 4.52 | 0.89 |
| 21.32 | 4.17 | 3.81 |
| 22.81 | 3.90 | 4.15 |
| 23.07 | 3.85 | 18.05 |
| 24.71 | 3.60 | 4.53 |
| 25.09 | 3.55 | 6.30 |
| 26.68 | 3.34 | 7.48 |
| 28.13 | 3.17 | 4.93 |
| 29.56 | 3.02 | 1.36 |
| 30.06 | 2.97 | 8.52 |
| 31.45 | 2.84 | 7.47 |
| 33.00 | 2.71 | 1.20 |
| 34.00 | 2.64 | 2.25 |
| 34.44 | 2.60 | 1.28 |
| 37.04 | 2.43 | 1.93 |
| 39.04 | 2.31 | 1.33 |

### Example 3 Preparation of Crystalline Form A

9.7 mg of Veliparib in powder was added into a 1.5 mL glass vial, followed by the addition of 0.5 mL of acetone. Then aqueous HCl solution (7.0 μL, 12 mol/mL) was dropped while stirring at room temperature. Keep stirring at room temperature for 12 hours. After being separated by centrifugation, the obtained solid was analyzed by XRPD, of which the crystal form was confirmed to be crystalline form A.

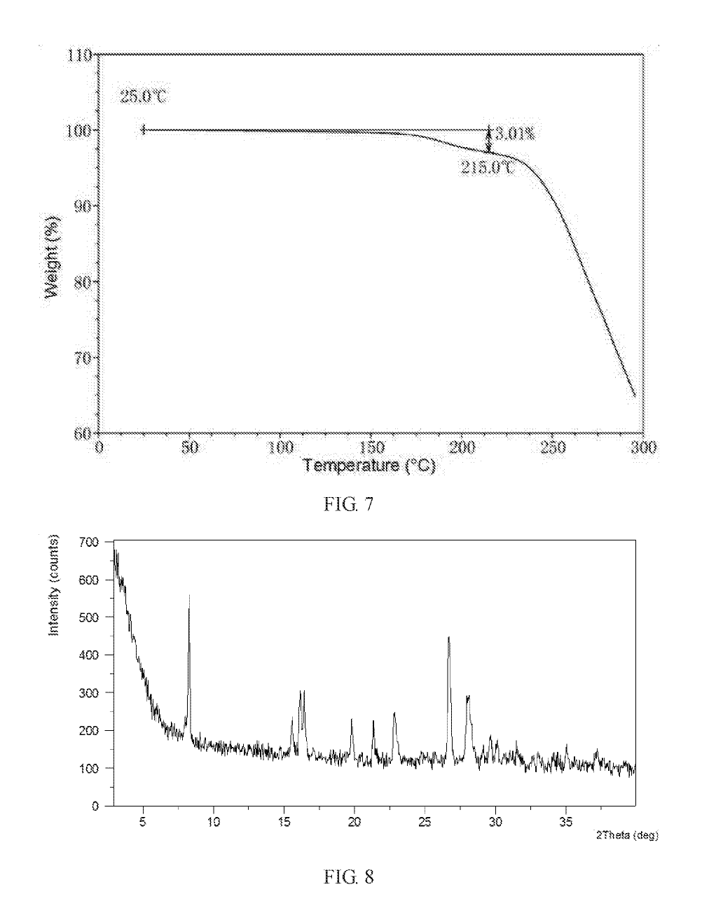

The XRPD data of Form A obtained in this example are listed in Table 3, and the corresponding XRPD pattern and TGA curve are displayed in FIG. 6 and FIG. 7, respectively.

TABLE 3

| 2theta | d-spacing | Intensity (%) |
|---|---|---|
| 8.27 | 10.69 | 100.00 |
| 15.55 | 5.70 | 20.07 |
| 16.16 | 5.48 | 19.85 |
| 16.43 | 5.39 | 18.75 |
| 19.79 | 4.49 | 13.47 |
| 21.32 | 4.17 | 15.62 |
| 22.82 | 3.90 | 15.57 |
| 23.08 | 3.85 | 14.47 |
| 24.71 | 3.60 | 4.75 |
| 25.10 | 3.55 | 7.49 |
| 26.71 | 3.34 | 37.62 |
| 28.12 | 3.17 | 20.84 |
| 29.64 | 3.01 | 7.27 |
| 30.08 | 2.97 | 12.37 |
| 31.48 | 2.84 | 5.36 |
| 33.00 | 2.71 | 6.59 |
| 34.21 | 2.62 | 2.30 |
| 37.05 | 2.43 | 4.28 |

### Example 4 Preparation of Form A

10.2 mg of Veliparib in powder was added into a 1.5 mL glass vial, followed by the addition of 0.5 mL of EtOAc. Then aqueous HCl solution (7.0 μL, 12 mol/mL) was dropped while stirring at room temperature. Keep stirring at room temperature for 12 hours. After being separated by centrifugation, the obtained solid was analyzed by XRPD, of which the crystal form was confirmed to be crystalline form A.

The XRPD data of crystalline form A obtained in this example are listed in Table 4, and the corresponding XRPD pattern is displayed in FIG. 8.

TABLE 4

| 2theta | d-spacing | Intensity (%) |
|---|---|---|
| 8.27 | 10.69 | 100.00 |
| 15.57 | 5.69 | 21.79 |
| 16.14 | 5.49 | 44.72 |
| 16.42 | 5.40 | 44.35 |
| 19.80 | 4.48 | 77.95 |
| 21.35 | 4.16 | 17.80 |
| 22.81 | 3.90 | 30.97 |
| 26.68 | 3.34 | 82.13 |
| 26.75 | 3.33 | 71.09 |
| 28.05 | 3.18 | 42.72 |
| 29.63 | 3.01 | 17.58 |
| 30.10 | 2.97 | 12.99 |
| 31.56 | 2.83 | 9.47 |
| 33.04 | 2.71 | 7.43 |
| 35.03 | 2.56 | 14.72 |
| 37.16 | 2.42 | 8.37 |

### Example 5 Particle Size Distribution Study

The particle size distributions (PSD) of crystalline form A and crystalline form 1 and form 2 in CN101821269B were studied. The PSD data are summarized in Table 5.

TABLE 5
PSD data

| Crystalline form | MV (μm) | D10 (μm) | D50 (μm) | D90 (μm) |
|---|---|---|---|---|
| Form A | 407.2 | 41.6 | 278.6 | 1118 |
| Form 1 | 20.89 | 11.54 | 18.89 | 31.97 |
| Form 2 | 94.50 | 8.99 | 59.08 | 231.9 |

Compared with the prior crystalline form 1 and form 2, crystalline form A has larger particle size. Generally, the large particle size of a drug makes it easy to filter and separate, thus saves time in process. Large particle size also has some effect on increasing the drug's stability. The drug with larger particle size has better flowability, which benefits the subsequent process.

### Example 6 Mechanical Stability Study

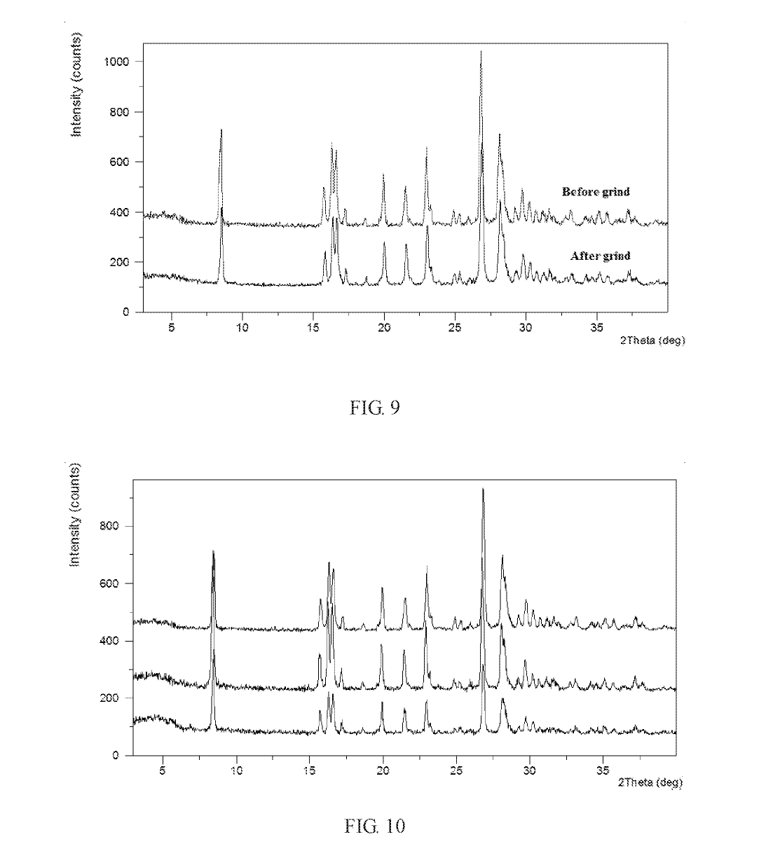

Crystalline form A of present disclosure was ground, and analyzed by XRPD before and after being ground for 5 minutes. The XRPD pattern was shown in FIG. 9. As is shown in FIG. 9, the XRPD patterns of crystalline form A show no significant change before and after being ground for 5 minutes, indicating good mechanical stability of crystalline form A.

### Example 7 Physical Stability Study

Crystalline form A was stored at 25° C./60% RH and 40° C./75% RH for one month. XRPD patterns before and after storage are shown in FIG. 10 and the corresponding results are summarized in Table 6.

TABLE 6

| Initial | Storage | | Crystalline |
|---|---|---|---|
| crystalline form | condition | Time | form change |
| Form A | 25° C./60% RH | 1 month | No change |
| Form A | 40° C./75% RH | 1 month | No change |

No form change was observed for crystalline form A after being stored at 25° C./60% RH and 40° C./75% RH for one month. The above results indicate that crystalline form A has good physical stability.

### Example 8 Solubility Study

Crystalline form A samples were dissolved into 1 mL of simulated gastric fluid (SGF), fed state simulated intestinal fluid (FeSSIF, pH 5.0), fasted state simulated intestinal fluid (FaSSIF, pH 6.5) and water to test the corresponding solubility. The results are shown in Table 7.

TABLE 7

| | SGF | FaSSIF | FeSSIF | Water |
|---|---|---|---|---|
| Weight/mg | 12.4 | 11.5 | 11.3 | 11.2 |
| Observation | Completely | Completely | Completely | Completely |
| | dissolved | dissolved | dissolved | dissolved |

As can be seen from the above results, crystalline form A of the present disclosure has excellent solubility in SGF, FaSSIF, FeSSIF and water. The solubility is higher than 10 mg/mL, which is at least 2 times higher than the crystalline forms disclosed in the prior art. Higher solubility can facilitate more efficient dissolution of API, reduce the amount of solvent, reduce energy consumption and environmental pressure in the process development, and improve drug's bioavailability.

The examples described above are only for illustrating the technical concepts and features of the present disclosure, and intended to make those skilled in the art being able to understand the present disclosure and thereby implement it, and should not be concluded to limit the protective scope of this disclosure. Any equivalent variations or modifications according to the spirit of the present disclosure should be covered by the protective scope of the present disclosure.

1. A crystalline form A of formula I compound dihydrochloride, 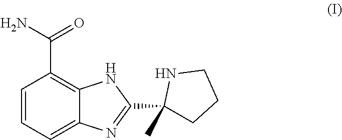 [SMILES: C[C@]1(c2nc3cccc(C(N)=O)c3[nH]2)CCCN1]
wherein the X-ray powder diffraction pattern shows characteristic peaks at 2theta values of 8.3°±0.2°, 26.7°±0.2°, and 16.1°±0.2°.

2. The crystalline form A according to claim 1, wherein the X-ray powder diffraction pattern further shows one or two or three characteristic peaks at 2theta values of 22.8°±0.2°, 21.3°±0.2°, and 15.6°±0.2°.

3. The crystalline form A according to claim 1, wherein the X-ray powder diffraction pattern further shows one or two characteristic peaks at 2theta values of 19.8°±0.2° and 28.1°±0.2°.

4. The crystalline form A according to claim 1, wherein the X-ray powder diffraction pattern further shows characteristic peaks at 2theta values of 22.8°±0.2°, 21.3°±0.2°, 15.6°±0.2°, 19.80°±0.2°, and 28.1°±0.2°.

5. A preparation method of crystalline form A of formula I compound dihydrochloride according to claim 1, wherein the method comprises:
1) adding formula I compound into a solvent system selected from alcohols, ketones, esters and nitriles, then stirring to react with hydrochloric acid to obtain a solid-liquid mixture system containing formula I compound dihydrochloride;
2) separating solid-liquid mixture system in step 1) to get the solid, and drying to obtain the crystalline form A.

6. The preparation method according to claim 5, wherein said alcohol is ethanol or isopropanol, and said ketone is acetone or methyl isobutyl ketone, and said ester is ethyl acetate or isopropyl acetate, and said nitrile is acetonitrile.

7. The preparation method according to claim 6, wherein said solvent system is one or more solvents selected from ethanol, isopropanol, acetone, methyl isobutyl ketone, ethyl acetate, isopropyl acetate, and acetonitrile.

8. The preparation method according to claim 5, wherein in step 1), formula I compound is added into the solvent system firstly, and then hydrochloric acid is added dropwise with stirring.

9. The preparation method according to claim 5, wherein a reaction temperature in step 1) is −20° C. to 50° C.

10. The preparation method according to claim 9, wherein the reaction temperature in step 1) is room temperature.

11. The preparation method according to claim 5, wherein in step 1), a concentration of said hydrochloric acid is 10-12 mol/L, and a molar ratio of said hydrochloric acid and formula I compound is 2-2.1:1.

12. A pharmaceutical composition, wherein the said pharmaceutical composition comprises a therapeutically effective amount of crystalline form A of formula I compound dihydrochloride according to claim 1 and pharmaceutical excipient.

13. A method of treating metastatic breast cancer, colon cancer, metastatic melanoma and brain tumors, comprising administering to a patient in need thereof a therapeutically effective amount of the crystalline form A of formula I compound dihydrochloride according to claim 1.

14. The preparation method according to claim 8, wherein a reaction temperature in step 1) is −20° C. to 50° C.

15. The preparation method according to claim 8, wherein in step 1), a concentration of said hydrochloric acid is 10-12 mol/L, and a molar ratio of said hydrochloric acid and formula I compound is 2-2.1:1.
